(19) United States
(12) Patent Application Publication
(10) Pub. No.: US 2025/0368941 A1
(43) Pub. Date: Dec. 4, 2025

(54) SURFACES WITH MODIFIED CELL ADHESION AND RELATED METHODS

(71) Applicant: Massachusetts Institute of Technology, Cambridge, MA (US)

(72) Inventors: Kripa K. Varanasi, Lexington, MA (US); Victor Leon, Cambridge, MA (US); Baptiste Blanc, Somerville, MA (US); Sophia Sonnert, Cambridge, MA (US)

(73) Assignee: Massachusetts Institute of Technology, Cambridge, MA (US)

(21) Appl. No.: 18/875,685

(22) PCT Filed: Jul. 11, 2023
(86) PCT No.: PCT/US2023/069978

§ 371 Date: Dec. 16, 2024

Related U.S. Application Data
(60) Provisional application No. 63/388,015, filed on Jul. 11, 2022.

(51) Int. Cl. C12M 1/42; C12N 13/00

(52) U.S. Cl. CPC C12M 35/02; C12N 13/00

(57) ABSTRACT

Surfaces in which the cell adhesion properties of the surface are modified by a change in surface potential are generally described.

### TECHNICAL FIELD

Surfaces in which the cell adhesion properties of the surface are modified by a change in surface potential are generally described.

### BACKGROUND

Controlling cell adhesion to surfaces is important, for example, for controlling cell growth and/or differentiation. However, it can be difficult to control the cell adhesion properties of cells without negatively impacting the cells themselves. Current methods to control adhesion rely on surface functionalization, which are typically cell specific and work by being toxic to some cells while non-toxic to other cells. However, toxicity to even some cells may be unacceptable in some conditions where cytotoxicity may limit compatibility with certain applications.

### SUMMARY

Surfaces that can modify cell adhesion properties are generally described. The subject matter of the present disclosure involves, in some cases, interrelated products, alternative solutions to a particular problem, and/or a plurality of different uses of one or more systems and/or articles.

In one aspect, a cell culturing system comprising a surface comprising a material having a dielectric constant of greater than or equal to 1.5 wherein the surface is configured to modify cell adhesion properties of the cell culturing system is described.

In another aspect, a cell culturing system comprising a surface comprising a material having a dielectric constant of greater than or equal to 1.5, wherein a surface charge of the surface is altered to modify cell adhesion properties of the cell culturing system is described. The material may comprise a non-polymeric material.

In a different aspect, a surface is described comprising a non-polymeric material having a dielectric constant of greater than or equal to 1.5, wherein the surface is configured to modify cell adhesion.

In another aspect, a surface is described comprising a material, wherein a breakdown voltage of the material is greater than or equal to 0.5 V, and power source in electric communication with the material, herein the power source is configured to provide a change in surface zeta potential of greater than or equal to 5 mV. The breakdown voltage may be Vₘₐₓ/t, where Vₘₐₓ is a maximum voltage applied to the polymeric material before damage to the material and where t is a thickness of the material or the surface.

In another aspect, a surface is described comprising a non-polymeric material, wherein a dielectric constant of non-polymeric material is greater than or equal to 20, and wherein a thickness of the surface is less than or equal to 1000 nm and/or greater than or equal to 0.1 nm.

In a different aspect, a method for modifying cell adhesion on a surface, the method comprising applying a voltage to the surface, wherein the surface has a dielectric constant of greater than or equal to 1.5; and depositing cells on the surface.

Other advantages and novel features of the present disclosure will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A is a schematic diagram of an unmodified surface with cells deposited, according to some embodiments;

FIG. 1B is a schematic diagram of a surface modified by applying a voltage in which less cells adhere to the surface relative to the unmodified surface, according to some embodiments;

FIG. 1C is a schematic diagram of a surface modified by applying a voltage in which more cells adhere to the surface relative to the unmodified surface, according to some embodiments;

FIG. 1D is a schematic diagram of a surface comprising a first portion, a second portion, and a third portion each with different cell adhesion properties relative to an adjacent portion, according to some embodiments;

FIG. 1E is a schematic diagram illustrating a surface that has been modified such that a closed shape on the surface is attractive towards cells while a portion outside the closed shape is repulsive towards cells, according to some embodiments;

FIG. 1F is a schematic diagram illustrating a surface that has been modified such that a closed shape on the surface is repulsive towards cells while a portion outside the closed shape is attractive towards cells, according to some embodiments;

FIG. 2A is a schematic of a setup used in some embodiments where a microfluidic of channel with a height of 30 μm and 6 mm width was loaded with algae and the algae were left to settle for 1 hour on a high-k dielectric with an 20 nm film deposited on a conductive surface, according to some embodiments;

FIG. 2B shows an image obtained via fluorescence microscopy showing the high adhesion of cells on polycarbonate, a material typically used to construct bioreactors relative to hafnia, shown in FIG. 2D, according to some embodiments;

FIG. 2C illustrates visible damage and breakdown across typical plastic, such as PET, induced by an applied electric field, according to one example;

FIG. 2D is an image obtained via fluorescence microscopy showing the relative low adhesion of cells on a hafnia surface, relative to the PC surface of FIG. 2B, with an applied negative potential, where adhesion is reduced on the hafnia surface, according to some embodiments;

FIG. 3A is schematic of the model demonstrating the relationship between the applied voltage (V) and change in zeta potential (ζ), where ϵ_{d}, ϵₗ, t_{d}, and t_{DL} correspond to dielectric permittivity, liquid medium permittivity, dielectric thickness, and double layer thickness, respectively, according to some embodiments (from which, in addition to other disclosure herein, provides sufficient direction to allow those of ordinary skill in the art to apply a variety of zeta potentials within a range suitable for use with this disclosure, at any one or a variety of surfaces or portions of surfaces);

FIG. 3B shows calculations of zeta potentials based on the capacitive theory for a 20 nm thick hafnia surface compared to a 20 nm thick polycarbonate surface, where hafnia enables access to a larger range of magnitudes as well as polarity switches, according to some embodiments;

FIG. 3C shows fluorescent microscope images of Chlorella vulgaris adhesion on hafnia with different applied voltages and, when the zeta potential was negative, reduced adhesion was observed, and, when the zeta potential was positive, increased adhesion was observed, according to some embodiments;

FIG. 4A shows cell adhesion against applied shear and applied voltage where the y-axis indicates the fraction of cells remaining from initial measurement of number of adhered cells surfaces before any shear is applied and where whisker plots of cell adhesion are shown and where each point shown is 1 measurement of a random surface area with 15 random surface areas are taken from 3 different experiments, with 5 area measurements taken from each and an “x” mark denoting the median point, according to some embodiments;

FIG. 4B shows cell adhesion against applied shear and applied voltage where the y-axis indicates the fraction of cells remaining from initial measurement of number of adhered cells surfaces before any shear is applied and where the fraction of remaining algae as a function of applied voltage and applied shear and where the background denotes where surface ζ is positive or negative, respectively, and, as ζ becomes more positive, an increase in cell adhesion is observed and where each point is from 15 random surface areas taken from 3 different experiments, with 5 area measurements taken from each experiment with error bars being the standard deviation of the, according to some embodiments;

FIG. 5A shows a fluorescent microscopy image with illustrating electrostatic repulsion by surface charging on a dielectric is shown with another dielectric, SiO₂, according to some embodiments;

FIG. 5B shows a fluorescent microscopy image illustrating that higher ionic concentration for saltwater algae (i.e., 0.6 M) screens the electrostatic effect, and although ζ changed sign, there was no observed effect on adhesion, according to some embodiments;

FIG. 5C shows a fluorescent microscopy image illustrating that longer duration experiments (i.e., 12 days) show that the electrostatic effect was robust, with cell culture and health appearing to be unaffected, according to some embodiments;

FIG. 6A shows the millimetric cell pattern, shaped like a tree, shown above has an attractive (+) region inside the pattern, where t_{d} was 10 nm and ζ was 12 mV where, in the repulsive (−) outer region, t_{d} was 40 nm and ζ was −12 mV, according to some embodiments;

FIG. 6B shows the millimetric cell pattern, shaped like a bear, shown above has an attractive (+) region, where t_{d} was 10 nm and ζ was 12 mV and where, in the repulsive (−) outer region, t_{d} was 40 nm and ζ was −12 mV, according to some embodiments; and

FIG. 6C shows the number of cells adhered in the attractive region appears to be twice as high as that in the repulsive region where data is taken from 5 random surface areas inside and outside of the two patterns after 4 weeks in algae culture medium, according to some embodiments.

### DETAILED DESCRIPTION

Articles and methods including surfaces with modified cell adhesion properties are generally described. The cell adhesion properties of conventional surfaces are typically modified using surface functionalizations that provide cytotoxicity to specific types of cells over other cells. However, cell toxicity is not always desired and surface functionalizations can be complicated by the need for cell specificity for each cell-type within a population of cells, making it challenging to provide a surface with general applicability to multiple cell types. Additionally, care has to be taken to ensure that the functionalization is not toxic to the desired cells while preventing the adhesion of other types of cells.

The Inventors have discovered that cell adhesion properties of a surface may be modulated by modifying the surface potential (e.g., as measured by the zeta potential) of the surface. For example, a voltage can be applied across a dielectric material (e.g., a dielectric film), changing the material's surface zeta potential (ζ). It has been recognized and discovered that a change in ζ (e.g., a change in polarity of ζ, a change in the magnitude of ζ) may modify the cell adhesion properties of the surface, for example, by allowing less cells to adhere relative to the unmodified surface. In some cases, the surface can be modified such that more cells adhere relative to the unmodified surface, and other cases still, the surface may be selectively modified such that certain portions (e.g., a first portion) of the surface have increased cell adhesion relative to the unmodified surface while other portions of the surface (e.g., a second portion) have decreased cell adhesion relative to the unmodified surface. In some such cases, the surface may also comprise another portion (e.g., a third portion) that is unmodified relative to other portions (e.g., a first portion, a second portion). Surfaces and related methods are described in more detail below.

Modifying the adhesion properties of a surface in this manner has several advantages over conventional surface modifications. Conventional surface modifications include, for example, surface patterning and/or surface coatings that provide selective cytotoxicity for some cells over other cells, as was mentioned above. However, these conventional surface modifications have certain drawbacks. Surface patterning, for example, may require complex surface lithography techniques and can be limited by the nanoscale dimensions typically required to achieve cell adhesion modification, making it difficult to scale. Surface coatings, on the other hand, are typically more scalable than surface patterning; however, these surface coatings can create contamination problems, as the surface coating requires the application of a chemical on the surface, and this chemical must be carefully selected to avoid cytotoxicity to the cells of interest. By contrast, modifying the surface charge of the surface, as described by this disclosure, avoids these drawbacks of conventional surface modifications, as no chemical coating is required, and no specialized patterning of the surface is needed to modify the cell adhesion properties of the surface. However, it will be understood that, for some embodiments, a chemical coating, specialized patterning, and/or the like can be used, if desired, in combination with surface charge modifications as described herein.

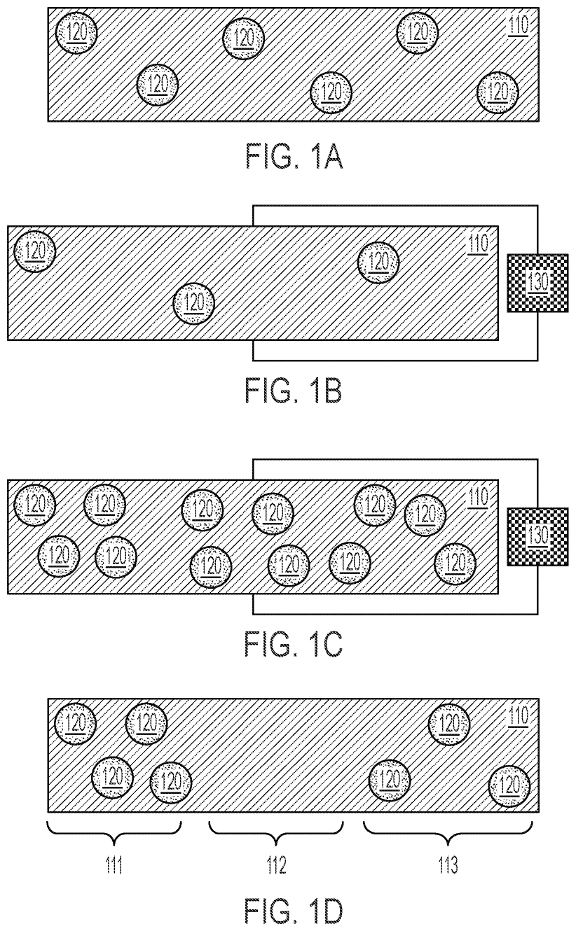

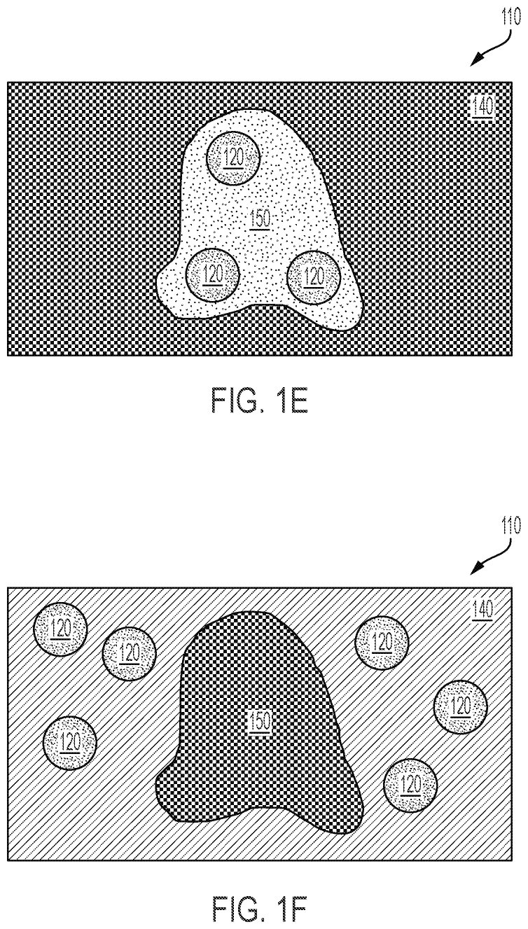

By way of illustration, FIGS. 1A-1D show non-limiting embodiments of surfaces with varying cell adhesion properties. For example, FIG. 1A is a schematic diagram of a surface 110 in which cells 120 have been deposited. The adhesion properties of the cells can be modified by altering or otherwise modifying the surface potential of the surface. For example, in FIG. 1B, a power source 130 provides a voltage across the surface, resulting in a modified zeta potential of the surface. As a result, the cell adhesion properties of the surface 110 in FIG. 1B have been modified relative to the surface in FIG. 1A. In FIG. 1B, the applied voltage results in less cells adhering to the surface 110 relative to FIG. 1A. Conversely, in FIG. 1C, the power source 130 provides a surface charge to surface 110 that results in more cells adhering to the surface 110 relative to the surface of FIG. 1A. In some embodiments, one or more power sources can provide one or more voltages such that the surface has multiple regions of varying cell adhesion. For example, in FIG. 1D, the surface has been modified such that a first portion 111, a second portion 112, and a third portion 113 have different surface cell adhesion properties. The first portion 111 has been modified such that there is more cell adhesion relative to the unmodified surface; the second portion 112 has been modified such that there is less cell adhesion relative to the unmodified surface; and the third portion 113 has not been modified relative to the unmodified surface and may have the same cell adhesion properties of the unmodified surface. One or more power sources may be associated with each portion to provide the same or different voltage as other portions (not shown in the figure). Details regarding power source, voltage, and potential are described in more detail elsewhere herein, and those skilled in the art will be capable in light of the present disclosure of selecting appropriate power sources and applying appropriate voltages for a desired cell adhesion application.

The surface can be modified such that some portions (e.g., a first portion) of the surface have an increased or decreased affinity for cells (e.g., increased adhesion, decreased adhesion) while other portions (e.g., a second portion, a third portion) of the surface have a decreased or increased affinity for the cells (e.g., decreased adhesion, increased). In this manner, a first portion of the surface may, for example, have an increased affinity for cells, while a second portion of the surface, outside of the first portion, may have a decreased affinity for the cells. By way of illustration, FIG. 1E shows an illustrative diagram where a closed shape 150 on the surface 110 has an increased affinity (e.g., an attractive force) for the cells 120. In this figure, the portions outside of the closed shape 150 (i.e., portion 140 of the surface 110) have a decreased affinity (e.g., a repulsive attraction) for the cells. However, it should be understood that other configurations are possible. For example, in FIG. 1F, the configuration illustrated in FIG. 1E is reversed, such that the closed shape 140 has a decreased affinity for the cells 120 while the portion of the surface 110 outside of the perimeter of the closed shape 150 (i.e., portion 140 of the surface 110) has an increased affinity for the cells 120. Of course, other configurations than those shown illustratively in FIGS. 1E-1F are possible, as this disclosure is not so limited, and those skilled in the art, in view of this disclosure, will be capable of configuring the surface (e.g., portions of the surface) to have desired levels of cell adhesion.

In some embodiments, the surfaces with modified cell adhesion properties may be within a cell-culturing system, such as a bioreactor. In some embodiments, the surface is configured to modify cell adhesion properties of the cell culturing system (e.g., increase cell adhesion of a portion of the cell culturing system, decrease cell adhesion of a portion of the cell culturing system).

As noted above, surfaces (e.g., a surface of a dielectric material) can be modified or otherwise altered to change the growth of cells (e.g., relative to a surface of the material that has not be modified). One or more modified surfaces can be included in a bioreactor or some other system for growing cells. Accordingly, the surface can be a surface of the bioreactor, and the bioreactor is configured to grow one or more cells (e.g., a first cell type, a second cell type). In some such embodiments, the bioreactor may be configured to grow one type of cell at a faster rate relative to another cell type, different from the other cell type.

In some embodiments, a bioreactor comprising one or more modified surfaces has a particular volume. In some embodiments, the volume of the bioreactor is greater than or equal to 1 mL, greater than or equal to 50 mL, greater than or equal to 100 mL, greater than or equal to 250 mL, greater than or equal to 500 mL, greater than or equal to 750 mL, greater than or equal to 1 L, greater than or equal to 1.5 L, greater than or equal to 2 L, greater than or equal to 3 L, greater than or equal to 5 L, greater than or equal to 10 L, or greater than or equal to 20 L. In some embodiments, the volume of the bioreactor is less than or equal to 20 L, less than or equal to 10 L, less than or equal to 5 L, less than or equal to 3 L, less than or equal to 2 L, less than or equal to 1.5 L, less than or equal to 1 L, less than or equal to 750 mL, less than or equal to 500 mL, less than or equal to 250 mL, less than or equal to 100 mL, less than or equal to 100 mL, less than or equal to 50 mL, or less than or equal to 1 mL. Combinations of the above-referenced ranges are also possible (e.g., greater than or equal to 1 mL and less than or equal to 20 L). Other ranges are possible as this disclosure is not so limited.

In some embodiments, the surface comprises a material (e.g., a non-polymeric material, an inorganic material, a ceramic material) having a particular dielectric constant. In some embodiments, the dielectric constant of the material is greater than or equal to 1.5, greater than or equal to 2, greater than or equal to 4, greater than or equal to 5, greater than or equal to 10, greater than or equal to 20, greater than or equal to 50, greater than or equal to 100, greater than or equal to 500, greater than or equal to 1,000, or greater than or equal to 2,000. In some embodiments, the dielectric constant of the material is less than or equal to 2,000, less than or equal to 1,000, less than or equal to 500, less than or equal to 100, less than or equal to 50, less than or equal to 20, less than or equal to 20, less than or equal to 10, less than or equal to 5, less than or equal to 4, less than or equal to 2, or less than or equal to 1.5. Combinations of the above-referenced ranges are also possible (e.g., greater than or equal to 1.5 and less than or equal to 2,000). Other ranges are possible this disclosure is not so limited.

The surface may comprise any suitable material that does not breakdown under a breakdown voltage. In some embodiments, the breakdown voltage is Vₘₐₓ/t, where Vₘₐₓ is a maximum voltage applied to the polymeric material before damage to the material and where t is a thickness of the material or the surface. Damage to the material can be determined using microscopy (e.g., fluorescence microscopy). In some embodiments, a breakdown voltage of the material is greater than or equal to 0.5 V, greater than or equal to 0.7 V, greater than or equal to 1 V, or greater than or equal to 1.5 V. In some embodiments, the breakdown voltage is less than or equal to 1.5 V, less than or equal to 1 V, less than or equal to 0.7 V, less than or equal to 0.5 V. Combinations of the foregoing are also possible (e.g., greater than or equal to 0.5 V and less than or equal to 1.5 V). Of course, other ranges are possible as this disclosure is not so limited.

In some embodiments, the surface comprises a non-polymeric material (e.g., an inorganic material, a ceramic material). In some such embodiments, the non-polymeric material is free of any organic material (e.g., free of organic polymers, such a polyethylene, polycarbonate). In some embodiments, the non-polymeric material comprises Sc₂O₃, Rb₂O₃, BeO, BaO, CaO, SrO, Si, SiO₂, Si₃N₄, Al₂O₃, Ta₂O₅, TiO₂, SrO, SrTiO₃, ZrO₂, HfO₂, HfSiO₄, La₂O₃, Y₂O₃, and/or LaAlO₃. In some embodiments, the material (e.g., the inorganic material) is electrically non-conductive (i.e., an insulating material).

In some embodiments, the surface comprises an inorganic material. In some embodiments, the surface comprises a metal oxide, such as hafnia (HfO₂) and/or silica (SiO₂).

The surface may have any suitable thickness. In some embodiments, the thickness of the surface (or a material of the surface) is greater than or equal to 0.1 nm, greater than or equal to 1 nm, greater than or equal to 5 nm, greater than or equal to 10 nm, greater than or equal to 25 nm, greater than or equal to 50 nm, greater than or equal to 100 nm, greater than or equal to 250 nm, greater than or equal to 500 nm, greater than or equal to 750 nm, or greater than or equal to 1,000 nm. In some embodiments, the thickness of the surface (or a material of the surface) is less than or equal to 1,000 nm, less than or equal to 750 nm, less than or equal 500 nm, less than or equal to 250 nm, less than or equal to 100 nm, less than or equal to 50 nm, less than or equal to 25 nm, less than or equal to 10 nm, less than or equal to 5 nm, less than or equal to 1 nm, or less than or equal to 0.1 nm. Combinations of the above-referenced ranges are also possible (e.g., less than or equal to 1,000 nm and greater than or equal to 0.1 nm). Other ranges are possible. In some such embodiments, the thickness of the material of the surface can, at least in part, control or modulate the cell adhesion properties of the surface.

In some embodiments, a surface charge of the surface is altered to modify cell adhesion properties of the cell culturing system. In some such embodiments, one or more power sources and/or one or more electrodes is attached to the surface and may provide a desired potential (e.g., a zeta potential), which may modify the cell adhesion properties of the cell culturing system (i.e., a surface of the cell culturing system).

In some embodiments, a surface charge of the surface is altered to modify cell adhesion properties of the cell culturing system. In some such embodiments, a power source is associated and/or in electric communication with the material with the surface and is configured to provide a change in a surface zeta potential of greater than or equal to 5 mV, for example, relative to the surface prior to applying the potential (e.g., an unmodified surface). In some embodiments, a power source is configured to provide a change in surface zeta potential of greater than or equal to 5 mV, greater than or equal to 10 mV, greater than or equal to 25 mV, greater than or equal to 50 mV, greater than or equal to 100 mV, greater than or equal to 250 mV, or greater than or equal to 500 mV. In some embodiments, a power source is configured to provide a change in surface zeta potential of less than or equal to 500 mV, less than or equal to 250 mV, less than or equal to 100 mV, less than or equal to 50 mV, less than or equal to 25 mV, less than or equal to 10 mV, or less than or equal to 5 mV. Combinations of the foregoing range are also possible (e.g., greater than or equal to 5 mV and less than or equal to 500 mV). Other ranges are possible.

In some embodiments, a surface is modified by applying a potential with a relatively low amount of power. In some embodiments, the power consumed while applying the potential is less than or equal to 1 mW, less than or equal to 750 μW, less than or equal to 500 μW, less than or equal to 250 μW, less than or equal to 100 μW, less than or equal to 50 μW, less than or equal 25 μW, less than or equal to 10 u W, less than or equal to 1 μW, less than or equal to 750 nW, less than or equal to 500 nW, less than or equal to 250 nW, less than or equal to 100 nW, less than or equal to 50 nW, less than or equal to 25 nW, less than or equal to 10 nW, or less than or equal to 1 nW. In some embodiments, the power consumed while applying the potential is greater than or equal to 1 nW, greater than or equal to 10 nW, greater than or equal to 25 nW, greater than or equal to 50 nW, greater than or equal to 100 nW, greater than or equal to 250 nW, greater than or equal to 500 nW, greater than or equal to 750 nW, greater than or equal to 1 μW, greater than or equal to 10 μW, greater than or equal to 25 μW, greater than or equal to 50 μW, greater than or equal to 100 μW, greater than or equal to 250 μW, greater than or equal to 500 μW, greater than or equal to 750 μW, greater than or equal to 1 mW. Combinations of the foregoing ranges are also possible (e.g., greater than or equal to 1 nW and less than or equal to 1 mW). Of course, other ranges are possible as this disclosure is not so limiting.

It should be understood that modifying the surface potential can be achieved in any of a variety of ways that would be understood to those of ordinary skill in the art. In one set of embodiments, a potential is applied across a section of a surface, e.g., by electrically biasing (e.g., by contacting with an electrode) one portion of the surface at one potential, and electrically connecting another portion of the surface with a different potential, creating a potential across the surface. In another set of embodiments, the surface is held at one potential, and an electrode is positioned to apply a potential difference between it and the surface, for example by being in contact with and electrically-conductive medium (e.g., an electrolyte) that is also in electrical contact with the surface. In some embodiments, the applied potential, in combination with the thickness of the surface (e.g., a thickness of a material of the surface) can modulate the cell adhesion properties of the surface. In some embodiments, the power source provides a positive voltage to the surface. In some embodiments, the power source provides a negative voltage to the surface. In some embodiments, the zeta potential of a surface is positive. In some embodiments, the zeta potential of a surface if negative. In some embodiments, the zeta potential of a surface is zero.

In some embodiments, an electrically conductive material is adjacent to the surface. In some such embodiments, the conductive material may also be in electric communication to the power source (e.g., via one or more wires).

In some embodiments, the surface is configured to adhere a particular amount of cells. Those skilled in the art, in view of this disclosure, will be capable of selecting appropriate materials and/or voltages in order to provide a particular degree of cell adhesion. In some embodiments, the surface is configured to adhere greater than or equal to 60 cells/mm², greater than or equal to 100 cells/mm², greater than or equal to 150 cells/mm², greater than or equal to 200 cells/mm², greater than or equal to 250 cells/mm², greater than or equal to 300 cells/mm², greater than or equal to 400 cells/mm², greater than or equal to 500 cells/mm², greater than or equal to 750 cells/mm², greater than or equal to 1,000 cells/mm², or greater than or equal to 1,100 cells/mm². In some embodiments, the surface is configured to adhere less than or equal to 1,100 cells/mm², less than or equal to 1,000 cells/mm², less than or equal to 750 cells/mm², less than or equal to 500 cells/mm², less than or equal to 400 cells/mm², less than or equal to 300 cells/mm², less than or equal to 250 cells/mm², less than or equal to 200 cells/mm², less than or equal to 150 cells/mm², less than or equal to 100 cells/mm², or less than or equal to 60 cells/mm². Combinations of the above-referenced ranges are also possible (e.g., greater than or equal to 60 cells/mm² and less than or equal to 1,100 cells/mm²). Other ranges are possible.

The surfaces described herein may be configured to adhere cells with a particular closed packed density. The closed packed density may describe the volume taken by number of particles in a given space of volume, and the particles may be approximated as discs as proxy for cell-shaped particles. In some embodiments, the surface is configured to adhere cells with a closed packed density of greater than or equal to 20%, greater than or equal to 50%, greater than or equal to 65%, greater treater than or equal to 80%. In some embodiments, the surface is configured to adhere cells with closed packed density of less than or equal to 80%, less than or equal to 65%, less than or equal to 50%, or less than or equal to 20%. Combinations of the above-referenced ranges are also possible (e.g., greater than or equal to 20% and less than or equal to 80%). Other ranges are possible. The packing density can be determined via fluorescence microscopy.

In some embodiments, one or more salts (e.g., NaCl) can be applied to one or more surface to further modify the cell adhesion properties of the surface (e.g., to decrease adhesion of cells on the surface).

For some embodiments, the surface can be modified to provide altered cell adhesion properties over a particular duration of time. For example, in some embodiments, the cell adhesion properties of the surface can be maintained for at least 1 second, at least 30 seconds, at least 1 minute, at least 1 hour, at least 3 hours, at least 6 hours, at least 12 hours, at least 18 hours, at least 1 day, at least 2 days, at least 3 days, at least 7 days, at least 1 week, at least 2 weeks, at least 4 weeks, at least 1 month, at least 3 months, at least 6 months, at least 12 months, or longer. Of course, other durations are possible and those skilled in the art, in view of this disclosure, will be capable of configuring a surface to have the desired cell adhesion properties for any suitable duration.

As noted above and elsewhere herein, the surfaces described herein may be suitable for a variety of applications, such as cell culturing applications. The surfaces can be modified as desired by a user (e.g., by controlling the surface zeta potential) to increase the adhesion of cells and/or decreased the adhesion of cells relative to an unmodified surface (e.g., with no potential applied across the surface). In some embodiments, the surface is configured to reduce cell adhesion. In some embodiments, the surface is configured to increase cell adhesion. In some embodiments, a surface has at least a first portion and a second portion with different cell adhesion properties (e.g., with different surface zeta potentials). In some embodiments, the surface within a bioreactor. In some embodiments, one or more modified surfaces is used in a water treatment process. In some embodiments, one or more surfaces may reduce biofouling (e.g., by suppressing the growth of biofouling bacteria relative to other bacteria). In one set of embodiments, the one or more surface (e.g., an exposed surface) is a part of a sensor. Other applications include, but are not limited to, medical implants, cell culturing apparatus such as petri dishes, photobioreactors, and tissue engineering.

The following examples are intended to illustrate certain embodiments of the present invention, but do not exemplify the full scope of the invention.

### EXAMPLE 1

The following example describes how cell adhesion properties of a surface can be affected by the application of a potential.

Controlling cell adhesion to surfaces is an important, but difficult, problem. Current methods to control adhesion rely on surface functionalization, which can be cell specific toxic to cells. By contrast, it has been discovered within the context of the present disclosure that cell adhesion may be modulated by using nanometric high-k dielectric films. A voltage can be applied across the dielectric film, changing the film surface's zeta potential, ζ. The use of high-performance dielectrics, HfO₂ and SiO₂, can change the ζ sign and magnitude over broad ranges of 100 mV by applying 1V across the dielectrics with 1 nW power draw. Freshwater chlorella vulgaris with a negative ζ were used as a model cell. It was demonstrated that cell adhesion can be inhibited when both surface and cell ζ are negative and generally enhanced when the surface ζ is positive and the cell ζ are negative. Toxicity to cells was generally limited, even after 4-week experiments. A systematic study of cell adhesion as a function of applied surface ζ and wall shear using a microfluidic chip was conducted. The importance of the electrostatic effect can be further shown with saltwater Nannochloropsis oculata. There were no observable changes in cell adhesion of N. oculata as a function of applied surface ζ, most likely due to screening of the electrostatic interaction by the more ionic culture medium of saltwater N. oculata (0.6 M) in comparison to freshwater C. vulgaris (0.01 M). Millimetric scale patterning experiments were also demonstrated.

Engineering cell adhesion to surfaces can be a difficult problem to solve and has important applications in medical implants, cell culture, photobioreactors, and tissue engineering. During mammalian cell culture, surfaces can be carefully prepared to increase cell adhesion by increasing surface hydrophobicity with plasma cleaning. Surface materials in algae photobioreactors can be selected to reduce biofouling on reactor walls. Many methods use a combination of surface texture and surface functionalizations to reduce cell adhesion. Surface functionalizations can be complicated and specific to each cell and surface type, making it challenging to provide a surface with general applicability to multiple cell types. Additionally, care has to be taken to ensure that the functionalization is not toxic to the cells.

The adhesion of a variety of cells to surfaces has been theoretically explained using classical and extended DLVO theory. The regime of DLVO interactions is longer range (1 nm to 10 nm) than biomolecules (<1 nm), which can be specific to each cell and surface type. As such, an engineered surface that can modulate the longer-range DLVO forces while limiting changes to the surface chemistry offers an enticing opportunity to lessen the specificity of biomolecular interactions and reduces the need to develop surface functionalizations.

The modulation of the electrostatic interaction in DLVO theory has been shown previously in microfluidic chips in the context of electro-osmosis and flow patterning. A surface's zeta potential (ζ), which is proportional to the electrostatic DLVO interaction, can be modulated by applying voltage across a dielectric. A low voltage and power engineered surface that modulates and influences cell adhesion using nanometric high-k dielectric films is reported. Freshwater algae Chlorella vulgaris and saltwater Nannochloropsis oculata were used as model cells. After applying 1 V across a dielectric film, cell adhesion was shown to be enhanced or inhibited by changing the magnitude and sign of a surface's ζ. This example describes cell adhesion when the electrostatic interaction between cell and surface is modulated rather than modulation via physico-chemical interactions. The dielectric materials tested, hafnia (HfO₂) and silicon dioxide (SiO₂), are considered transparent and non-toxic to cells. Moreover, a long term 2 and 4 week adhesion experiment was conducted with millimetric scale patterns to demonstrate that the effect is robust and operates for extended durations.

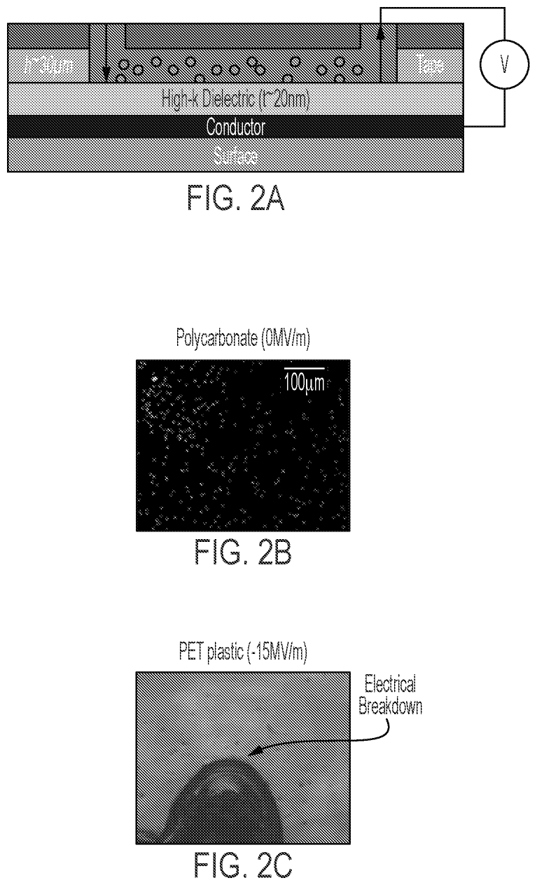

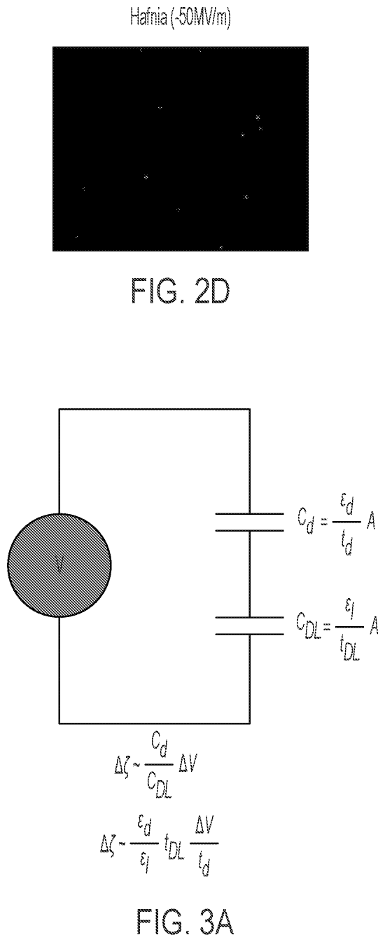

A schematic of a microfluidic setup used to collect data is shown in Error! Reference source not found.A. Using a particular channel geometry, and a particular flowrate and wall shear stress was applied to the cells. Since the model cell is algae, the technology was demonstrated in the context of algae photobioreactors, in which lower adhesion is desired to minimize the cleaning internal reactor walls. Polycarbonate, which has relatively high adhesion (FIG. 2B), is typically used to make photobioreactor walls. However, an applied high voltage across this polycarbonate plastic may end in catastrophic breakdown before any notable effect on adhesion can be observed (FIG. 2C). Using hafnia, a larger negative voltage can be applied to the surface and less adhesion is observed (FIG. 2D).

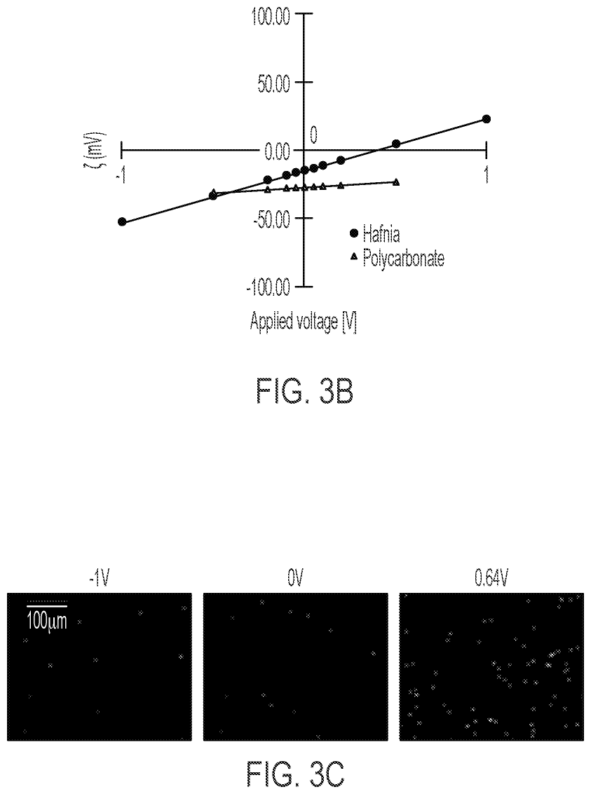

The effect of changing surface ζ and its influence on the electrostatic DLVO interaction between surface and cell, by applying voltage, V, can be rationalized with a simple capacitor model. The double layer and dielectric are modeled as two capacitors in series (FIG. 3A). Δζ˜ΔV(C_{d}/C_{DL}), where ΔV, C_{d}, and C_{DL} are the voltage applied across the dielectric, capacitance of the dielectric layer, and capacitance of the double layer, respectively. Capacitance is defined as C=A(ϵ/t), where A, ϵ, and t are the surface area, relative permittivity, and layer thickness, respectively. From this capacitive model, it is observed that a dielectric layer with large ϵ_{d} and small t_{d} is considered desirable. Another parameter for the selection of the dielectric material in the system is the electric breakdown field strength, E_{BD}=Vₘₐₓ/t. A larger E_{BD}, which generally enables larger magnitudes of ΔVₘₐₓ and Δζₘₐₓ.

Note that, since Δζ˜C_{d}/C_{DL}˜ϵ_{d}/ϵₗ, a larger dielectric permittivity, ϵ_{d}, relative to ϵₗ, the permittivity of the liquid, is considered desirable. Compared to the usual cell culture medium, water (ϵₗ˜80), most materials have low ϵ_{d}˜1. For example, common plastics, like PET and polycarbonate, have ϵ_{d}˜4. Therefore, hafnia (ϵ_{d} of 20) may be considered a high-k, or high permittivity, material. In the system, a hafnia layer with an E_{BD} of 50 MV/m was deposited with a t_{d} of 20 nm via atomic layer deposition. The liquid double layer had a t_{DL} of 3 nm for C. vulgaris' freshwater (c of 0.01 M) MB3N medium as calculated from the relationship for Debye length κ⁻¹=t_{DL}=˜0.304/√c nm.

A combination of high-k dielectric, nanometric thickness, and large E_{BD} allowed the system to enter a regime wherein ζ at the surface of the dielectric facing the cell can be changed from positive to negative, and the magnitude of ζ can be modulated in a 100 mV range (FIG. 3B). In FIG. 3B, polycarbonate had a much smaller possible range of ζ and was unable to flip its sign before catastrophic breakdown, as shown in FIG. 2C. Applying Vₘₐₓ up to E_{BD} across plastics seemed to have had no effect on cell adhesion.

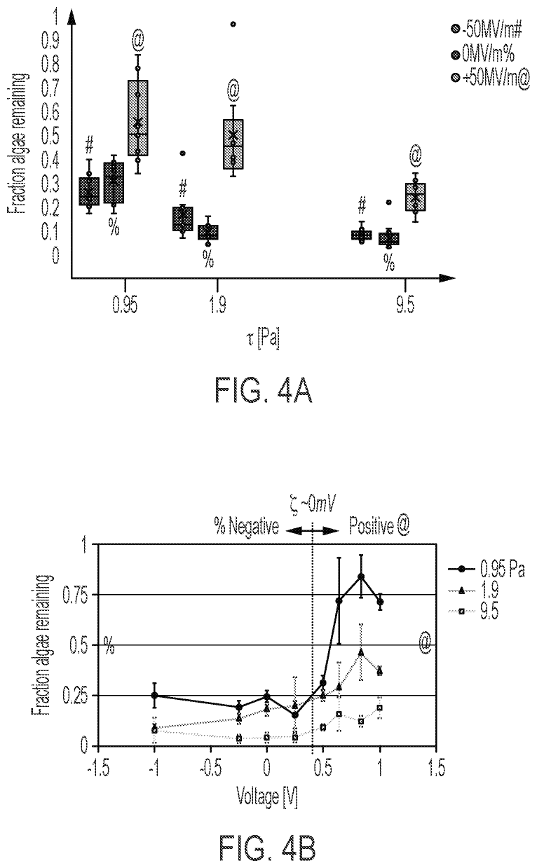

In FIG. 3C, a modulated voltage was applied, and the adhesion of algae cells on hafnia surfaces was measured. FIG. 4A and FIG. 4B show a systematic study on the effect of varying voltage and shear applied. Cells were allowed to settle for 1 hour and each point in the plots were from 3 separate experiments, with 5 random area samples per experiment for a total of 15 random areas per point in each plot. An 1 V applied voltage resulted in a˜1 nW power draw. The polarity of the surface ζ had an effect on cell adhesion. When ζ was negative, cell adhesion was mostly inhibited. When ζ was positive, cell adhesion was enhanced. The magnitude of negative ζ did not appear to have had an effect on further reducing adhesion, while, for positive ζ, the magnitude of ζ seemed to cause an increase in adhesion strength. The shear forces required to remove settled algae were comparable in literature at ˜1 Pa.

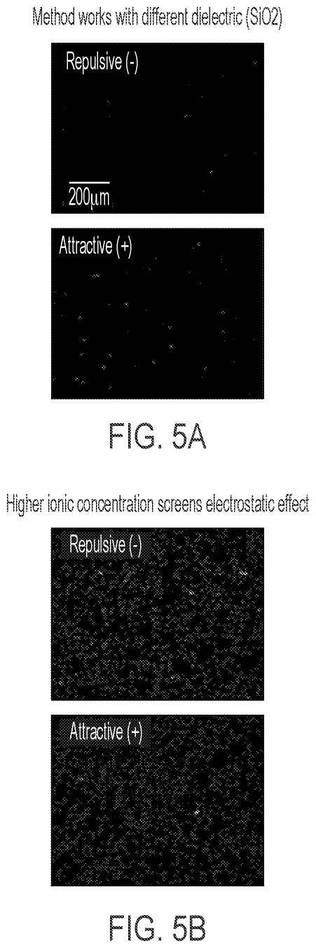

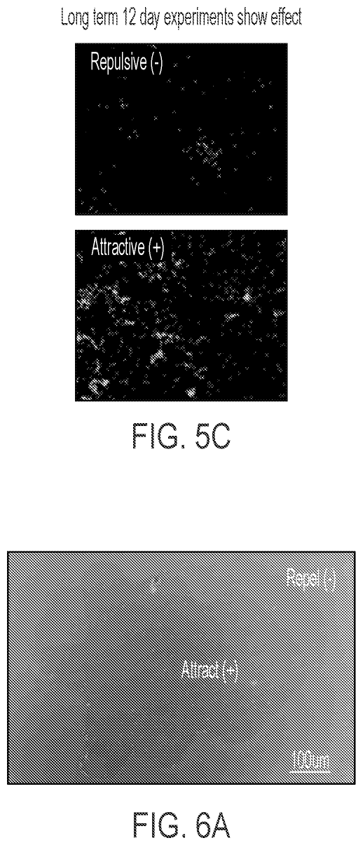

The versatility of the technique as applied to other materials was shown in FIG. 5A where a negative and positive ζ was applied on SiO₂ instead of hafnia, and an effect of decreasing and increasing adhesion, respectively, was demonstrated. Although SiO₂, in some cases, may not be considered a high-k dielectric (ϵ_{SiO2}˜4 vs ϵ_{HfO2}˜20), it is considered overall a high-performance dielectric due to its high E_{BD} of 890 MV/m, which is higher than hafnia's E_{BD} of 50 MV/m. This may allow for a range of accessible ζ and the ability to change the sign of surface ζ.

Since the phenomenon depends on the electrostatic interaction between cell and surface, higher salt concentrations may screen the electrostatic interaction, thus lessening the effect. This was demonstrated with saltwater Nannochloropsis oculata, which were cultured in Erdschreiber's medium (0.6 M). Erdschreiber's medium has a higher ionic concentration than the C. vulgaris' freshwater MB3N medium (0.01 M) used as noted above. FIG. 5B shows that there was limited effect on changing the sign of ζ in the saltwater medium, further validating that the electrostatic interactions are influential on the phenomenon and may screen the surface. Longer term experiments of 12 days (FIG. 5C) with hafnia and C. vulgaris demonstrated that the repulsive and attractive effect remained. The algae continue to adhere over the course of 12 days, and the algae continue to culture and grow in the test beaker.

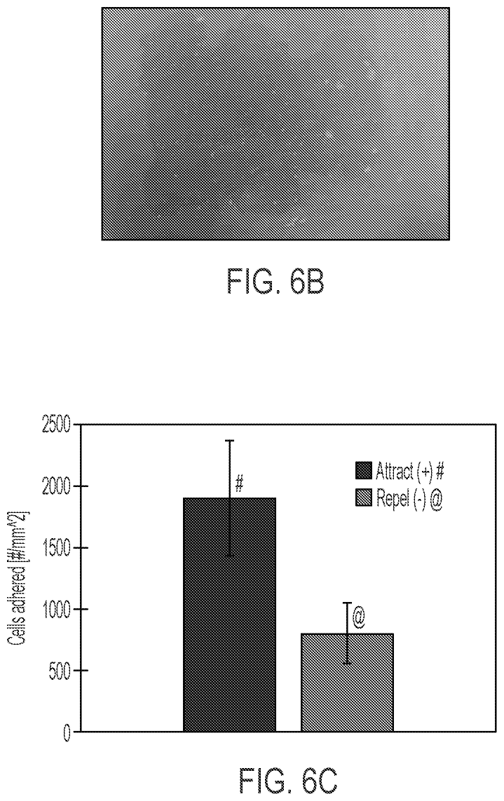

The ability to pattern cells on the surface was demonstrated by patterning the thickness of the hafnia layer. Since Δζ˜ΔV/t_{d}, at the same applied voltage, there will be a different Δζ at locations with different t_{d}. With an appropriate selection of thickness, inversions of surface ζ are possible. In FIG. 6A and FIG. 6B, a millimetric-sized tree and bear patterned surface are shown, where 0.43 V had been applied. Measurements were taken after the algae settle for 4 weeks on patterned glass slides in a beaker. Inside the patterns, the hafnia film had a thickness of 10 nm, which resulted in ζ₊ of 12 mV. Outside, the hafnia film had a thickness of 40 nm resulting inζ₋ of 12 mV. Measurements of 5 random areas, shown in FIG. 6C, in the inner (attractive) and outer (repulsive) reveal that adhesion in the ζ₊ regions were double the adhesion in the ζ₋ regions. Again, over the 4 weeks, there was no noticeable effect on the algae's growth and culture.

This example demonstrated that a low voltage and power (1 V, 1 nW) engineered films can modulate cell adhesion. The system worked by modulating the surface ζ, which thereby changed the electrostatic DLVO interaction between the surface and cells. The importance of the electrostatic effect was further shown through experiments with saltwater algae (0.6 M), where the higher ionic screening of the electrostatic effect potentially limited the effect of changing ζ. Using algae C. vulgaris, which have negative ζ, algae adhesion is enhanced when surface ζ is positive and reduced when ζ is negative. In contrast to previous approaches, this method did not exclusively rely on a chemical change of the interface. Furthermore, the method works with hafnia and SiO₂ which is considered non-toxic and transparent. Long term, 2-week experiments demonstrated that the effect is robust. After 1-month patterning experiments, algae cells continued to culture and grow. With further development, nano-engineered dielectric surfaces could be used for controlling adhesion in cell culture, tissue engineering, and photobioreactors. Further probing of different cells at intermediate ionic concentrations may be a fruitful direction of research. The consideration of other cell types and mediums would provide valuable information on how different cell types interact electrostatically with surfaces.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements.

This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A cell culturing system, comprising:
a surface comprising a material having a dielectric constant of greater than or equal to 1.5;
wherein the surface is configured to modify cell adhesion properties of the cell culturing system.

2. A cell culturing system as in claim 1,
wherein a surface charge of the surface is altered to modify the cell adhesion properties of the cell culturing system.

3. The cell culturing system of claim 1, wherein the material comprises a non-polymeric material.

4. A surface, comprising:
a non-polymeric material having a dielectric constant of greater than or equal to 1.5,
wherein the surface is configured to modify cell adhesion.

5. A surface, comprising:
a material, wherein a breakdown voltage of the material is greater than or equal to 0.5 V, and
a power source in electric communication with the material,
wherein the power source is configured to provide a change in surface zeta potential of greater than or equal to 5 mV.

6. The surface of claim 5, wherein the breakdown voltage is Vₘₐₓ/t, where Vmax is a maximum voltage applied to the polymeric material before damage to the material and where t is a thickness of the material or the surface.

7. The surface of claim 4, wherein the non-polymeric material
has a dielectric constant greater than or equal to 20, and
a thickness of the surface is less than or equal to 1000 nm and/or greater than or equal to 0.1 nm.

8. The cell culturing system of claim 1, wherein the surface is configured to adhere greater than or equal to 60 cells/mm² and/or less than or equal to 1,100 cell/mm².

9. The cell culturing system of claim 1, wherein the surface is configured to adhere cells with a closed packed density of greater than or equal to 20% and/or less than or equal to 80%.

10. The cell culturing system of claim 4, wherein a breakdown voltage of the non-polymeric material is greater than or equal to 1.5 V.

11. The cell culturing system of claim 1, wherein a dielectric constant of the material or the non polymeric material is greater than or equal to 20.

12. The cell culturing system of claim 1, wherein the surface is configured to reduce cell adhesion.

13. The cell culturing system of claim 1, wherein the surface is configured to increase cell adhesion.

14. The cell culturing system of claim 1, wherein the material is free of organic material.

15. The cell culturing system of claim 1, wherein the material comprises an inorganic material.

16. The cell culturing system of claim 1, wherein the material comprises a metal oxide.

17. The cell culturing system of claim 4, wherein the non-polymeric material comprises Sc₂O₃, Rb₂O₃, BeO, BaO, CaO, SrO, Si, SiO₂, Si₃N₄, Al₂O₃, Ta₂O₅, TiO₂, SrO, SrTiO₃, ZrO₂, HfO₂, HfSiO₄, La₂O₃, Y₂O₃, and/or LaAlO₃.

18. A method for modifying cell adhesion on a surface, the method comprising:
applying a voltage to the surface, wherein the surface has a dielectric constant of greater than or equal to 1.5; and
depositing cells on the surface.

19. The method of claim 18, further comprising changing or modifying a surface zeta potential of the surface by greater than or equal to 5 mV.

20. The method of claim 18, further comprising applying a positive voltage to the surface.

21. The method of claim 18, further comprising applying a negative voltage to the surface.

22. The method of claim 18, wherein greater than or equal to 20% of a closed packed density of the surface adheres cells and/or less than or equal to 80% of a closed packed density of the surface adheres cells.
